(19) United States
(12) Patent Application Publication
(10) Pub. No.: US 2009/0175783 A1
(43) Pub. Date: Jul. 9, 2009

(54) COMPOUNDS AND IMAGING METHODS

(76) Inventors: Jose M. Zubeldia, London (GB); Massimo Pistolesi, Florence (IT); Vijay V. Kakkar, London (GB); Simon Roitt, Hertfordshire (GB); Brian McParland, Buckinghamshire (GB); Diane Rickwood, Buckinghamshire (GB)

Correspondence Address: GE HEALTHCARE, INC., IP DEPARTMENT, 101 CARNEGIE CENTER, PRINCETON, NJ, 08540-6231, US

(21) Appl. No.: 11/997,336

(22) PCT Filed: Aug. 3, 2006
(86) PCT No.: PCT/GB06/02889

§ 371 Date: Oct. 14, 2008

(30) Foreign Application Priority Data
Aug. 3, 2005 (GB) 0515974.4

(51) Int. Cl. A61K 51/00; A61K 49/00; A61B 5/055

(52) U.S. Cl. 424/1.11; 424/9.1; 424/9.3

(57) ABSTRACT

The present invention relates to methods of distinguishing between active and inactive blood clots in vivo, including assisting in the determination of whether it is appropriate or not to continue anticoagulant therapy for an individual patient with previously diagnosed venous thrombo-embolic disease (VTE).

### FIELD OF THE INVENTION

The present invention relates to methods of distinguishing between active and inactive (ie. stable or resolving) clots in vivo to support a patient management decision. In particular, the invention provides assistance in the determination of whether it is appropriate or not to continue anticoagulant therapy for an individual patient with previously diagnosed venous thrombo-embolic disease (VTE).

### BACKGROUND TO THE INVENTION

Venous thrombo-embolic disease (VTE), which comprises deep vein thrombosis (DVT) and pulmonary embolism (PE), is an extremely common condition yet much remains to be learnt about the underlying mechanisms and tailoring treatment to the individual patient depending on individual risk factors [Lopez et al, Hematology, 439-456 (2004)].

About 2 million cases of suspected deep vein thrombosis (DVT) and 600,000 cases of suspected pulmonary embolism (PE) are diagnosed annually in the USA. These diagnoses are confirmed for 250 to 350 k DVT cases and about 60,000 PE subjects in the USA per annum. PE is the most common cause of in-hospital death in patients older than 65. The conventional treatment is anticoagulation therapy, typically a 5 to 10 day course of heparin followed by up to 3 to 12 months of warfarin. Anticoagulation inhibits further thrombus deposition, permits established thrombi to undergo stabilisation and/or endogenous lysis, and reduces the risk of recurrence of thrombosis [Kearon, Circulation, 107, 1-22 to I-30 (2003)]. The decision on whether to continue oral anticoagulation or not is currently made solely on clinical grounds.

There are no universally agreed guidelines in the medical community. The risk of recurrent VTE in the first 12 months following cessation of anticoagulation treatment depends on a complex range of risk factors associated with the initial VTE episode, but three groups of patients can be identified [Kearon, Circulation, 110, I-10 to I-18 (2004)]:

(i) low risk (ca. 3% recurrence)—major reversible risk factor (eg. surgery) identified as the sole explanation for the VTE;
(ii) intermediate risk (ca. 5% recurrence)—patients with VTE provoked by a reversible risk factor (eg. leg trauma, estrogen therapy or prolonged air travel);
(iii) high risk (ca. 10% recurrence)—patients with unprovoked (“idiopathic”) VTE and with persistent, irreversible or other risk factors such as cancer.
The present inventors would add to the high risk group patients having a genetic predisposition to thrombosis.

Recurrent VTE is an important risk factor for death after PE and for venous stasis syndrome after DVT and is associated with significantly increased long term healthcare and welfare costs (eg £500M in the UK in 2004). The optimal duration of anticoagulation treatment is not defined. Guidelines (ACCP2004) provide durations of treatment based on a patient's underlying risk factors and tend to err on the side of caution. For some patients a three month course of treatment appears sufficient, whereas for others, prolonged anticoagulation is viewed as necessary to reduce the likelihood of recurrent DVT. Prolonged anticoagulation is, however accompanied by an increased risk of major bleeding. Prolonged anticoagulation therapy in those patients who might benefit from a shorter course of treatment, may expose such patients to unnecessary risk. On the other hand, premature cessation of anticoagulation therapy in those patients with a real prospect of recurrence of DVT exposes those patients to unnecessary risk also. Thus, approximately 10 to 15% of patients with acute DVT of idiopathic origin or associated with ongoing risk factors develop recurrent acute DVT within 3 months following cessation of anticoagulation therapy. This implies an increased medical risk for the patient as well as additional costs for the health service provider. Kearon et al [Circulation, 110, I-10 to I-18 (2004)] also propose guidelines on the duration of anticoagulant therapy based on the patient's risk factors for VTE, together with their possible risk of bleeding complications.

There is therefore a need for an objective means of determining whether an individual patient with known active VTE disease still has sites of active thrombosis or not (the subject's disease status) and ultimately whether it is both appropriate and safe to cease anticoagulation therapy for that individual patient.

### THE PRESENT INVENTION

The present invention provides a method of distinguishing between active and inactive thrombus in vivo in an individual patient previously diagnosed with one or more known locations of venous thrombo-embolism (VTE) by virtue of its ability to label FXIIIa, a transglutaminase responsible for linking fibrin during clot formation.

Blood clots are composed of significant amounts of fibrin, which is cross-linked and stabilised by the action of the transglutaminase Factor XIIIa. Pulmonary emboli (PE) arise mostly from proximal deep vein thrombosis of the lower extremities. Both fibrin and Factor XIIIa are generated from non-active precursors specifically at sites of thrombosis. Peptide fragments of α2-antiplasmin are potent substrates for Factor XIIIa, and are thus covalently bound to the fibrin within active thrombi via the action of this enzyme.

Clotting is associated with active disease manifested by inflammation which results in the release of factors which promote clot formation. An actively forming thrombus is formed in response to trauma (either external or internal) whether physical (infectious agent or vascular damage) or pathology (disease process). In the context of VTE, the presence of an actively forming clot is associated with an ongoing disease pathology that may require treatment. Mediators of inflammation which promote clotting, elevating levels of FXIIIa which promotes the cross-linking of fibrin, predominate during clot formation. Once the source of the trauma has been isolated or removed, factors promoting healing predominate. Thus, an active clot signifies an ongoing disease or pathology which requires treatment (typically continuous anticoagulation therapy), whereas an inactive or resolving clot indicates that the cause of the original trauma has been dealt with indicating that the need for intervention is diminished.

The present invention also provides an objective method of determining whether anticoagulant therapy can be ceased for an individual patient suffering from VTE.

### DETAILED DESCRIPTION OF THE INVENTION

In a first aspect, the present invention provides a method of determining whether or not it is appropriate to cease anticoagulation therapy for an individual patient previously diagnosed with venous thrombo-embolism (VTE) who is considered to be potentially ready for cessation of anticoagulant therapy, which method comprises:
(i) imaging said patient with a thrombus imaging agent which comprises a compound which accumulates at sites of actively forming thrombosis in vivo labelled with an imaging moiety suitable for external imaging of the human body;
(ii) making a determination from the imaging of step (i) whether there is abnormal uptake of the thrombus imaging agent at the known locations of VTE relative to venous tissue adjacent to the site of VTE;
(iii) when the determination of step (ii) shows abnormal uptake, that site of thrombosis is identified as being active and the anticoagulant drug therapy for that patient is continued;
(iv) when the determination of step (ii) is normal, that site of thrombosis is identified as being inactive and the anticoagulant drug therapy for that patient is ceased.

By the phrase “whether or not it is appropriate to cease anticoagulation therapy” is meant a determination of whether or not cessation is in the best interests of the patient because, for that individual, the risk benefit ratio of taking the medication might be different from that when the patient started taking the medication, as a result of the evolution of the disease when treated.

By the phrase “previously diagnosed with venous thrombo-embolism (VTE)” is meant that the patient has already been positively diagnosed with VTE and preferably the location of the site(s) of VTE in that individual patient are known. This diagnosis can be carried out by methods known in the art which simply show the presence of sites of VTE at defined locations in vivo, without distinguishing between active (those requiring treatment) and inactive thrombi (those where treatment could cease). Such methods include clinical assessment, contrast venography, CT scanning, venous ultrasonography, pulmonary angiography, D-dimer assay (D-dimer is a plasmin-derived degradation product of crosslinked fibrin) and radiopharmaceutical imaging (eg. ventilation perfusion or V/Q scans) and combinations thereof. These have been reviewed by Lee et al [Ann. Rev. Med., 53, 15-33 (2002)], Sostman [Quart. J. Nucl. Med., 45, 311-323 (2001)] and Taillefer [Semin. Nucl. Med., 31, 102-123 (2001)]. The previous diagnosis is preferably carried out by in vivo imaging using a “thrombus imaging agent”. Such “thrombus imaging agents” encompass “active thrombus imaging agents” as defined below, as well as imaging agents which are taken up in both active and inactive thrombi, and hence do not differentiate between them. Examples of such non-differentiating thrombus imaging agents include:
(i) anti-fibrin antibodies or fragments thereof such as those taught by Bautovich et al [J. Nucl Med., 35(2) 195-202 (1994)] and Knight [Nucl. Med. Comm., 9(10), 823-829 (1988)];
(ii) labelled platelets;
(iii) labelled anti-platelet antibodies;
(iv) labelled RGD peptides (which target the products of clot breakdown which exist throughout a clot's ‘lifecycle’).

The thrombus imaging agent is preferably suitable for MRI, near infra-red (NIR) or radiopharmaceutical imaging, and is most preferably a radiopharmaceutical. The thrombus imaging agent is preferably an “active thrombus imaging agent” which comprises a “compound which accumulate at sites of active thrombus formation in vivo” as defined below. This means that comparative imaging is easier, because the background uptake is similar, permitting easier comparisons and image subtraction techniques.

By the term “potentially ready for cessation of anticoagulant therapy” is meant that the patient has reached the end of their anticipated minimum treatment period (defined by guidelines such as American Heart Association and British Thoracic Society) and has not experienced further venous thrombotic events or other clinical signs of disease in conjunction, and an assessment of the patient's underlying risk factors is also considered suitable for cessation of anticoagulation therapy. Patients with idiopathic venous thrombosis having been treated for at least 3 to 6 months are most suitable, principally because the correct period of treatment is not tightly defined. Treatment guidelines such as those from the ACCP (2004) are guidelines and by nature conservative. Although some patients might remain on treatment for many months or years, stopping anticoagulant therapy is often desirable due to the increased risk of major bleeding complications. Even patients with cancer (a major risk factor for VTE) are considered for treatment cessation at some point. Patients are often kept on long term therapy for cautionary reasons rather than an empirically based treatment decision.

By the term “anticoagulation therapy” is meant patient treatments which are designed to inhibit the process of coagulation of blood, ie. that inhibit effectively the coagulation cascade in one or more than one of its components. Suitable such medication may be administered either orally or intravenously. Intravenous agents are typically used for short term courses of therapy, especially when the patient is hospitalised, of duration up to 7-10 days in accordance with treatment guidelines. Suitable intravenous agents are chosen from: heparin or low molecular weight fractionated heparin (LMWH). Suitable oral agents are chosen from a vitamin K inhibitor or antagonist or enoxaparin. A preferred vitamin K inhibitor or antagonist comprises warfarin. These and other anticoagulant therapies, including newer drugs for this treatment, are described by Hyers [Arch. Intern. Med. Rev., 163, 759-768 (2003)].

By the term “active thrombus” is meant a clot that has levels of FXIIIa that promote the cross-linking of fibrin and subsequent clot formation resulting in abnormally increased thrombotic activity representing a risk for further events.

By the term “inactive thrombus” is meant a clot with no underlying active abnormal clotting process which hence does not represent a risk for further progression of the VTE. Such inactive clots may be either stabilised (ie. a structure which is not developing further or at risk of breaking off into the patient's bloodstream), or resolving (ie. being naturally broken down by the patient's metabolism).

By the term “active thrombosis imaging agent” is meant an imaging agent which can discriminate between active and inactive thrombi (as defined above) in vivo, by exhibiting selective uptake at sites of active thrombosis and little or no uptake relative to background at locations of stabilised thrombus.

By the term “compound which accumulates at sites of active thrombosis in vivo” is meant a compound which is covalently linked or crosslinked into the thrombus by the action of blood clotting factors or agents such as Factor XIIIa. Examples of such compounds include:
(i) fibrin-binding peptides based on the fibrin binding domain of fibronectin such as those taught by Ezov et al [Thromb. Haemost., 77(4), 796-803 (1997)] and Botnar et al [Circulation, 109(16), 2023-2029 (2004)];
(ii) peptide fragments of antiplasmin, such as those described by Jaffer et al [Circulation, 110, 170-176 (2004)], which are crosslinked into the active thrombus by Factor XIIIa;
(iii) agents which target the IIIa/IIb receptor associated with sensitised platelets which coalesce during the early phase of thrombus formation, such as Acutect™, which is a ^{99m}Tc radiopharmaceutical [Bernaducci Surg. Technol. Int., 12, 50-67 (2004) and J. Nucl. Med., 47(4) 292-320 (2003)];

The compound which accumulates at sites of active thrombosis in vivo may be of synthetic or natural origin, but is preferably synthetic.

Step (ii) of the method of the present invention comprises determining from the imaging of step (i) whether there is abnormal uptake of the thrombus imaging agent at the known locations of VTE relative to venous tissue adjacent to the site of VTE. Such “abnormal uptake” could manifest itself as either increased or decreased uptake relative to venous tissue adjacent to the site of VTE. When the active thrombus imaging agent comprises a compound which is actively covalently bonded to the clot by the action of blood clotting factors or agents, this is expected to manifest itself as increased uptake relative to the surrounding background.

Suitable thrombosis targeting peptides of the present invention are 3-80 mer peptides (ie. peptides comprising 3 to 80 amino acids), preferably 4 to 50-mer, most preferably 5 to 30-mer such fragments. The peptides may be cyclic or linear or combinations thereof. The peptides may be of synthetic or natural origin, but are preferably synthetic. The amino acid sequences of α₂-antiplasmin and fibronectin can be found in the following references: α₂-antiplasmin precursor [M. Tone et al., J. Biochem, 102, 1033, (1987)]; fibronectin [A. Gutman et al, FEBS Lett., 207, 145, (1996)].

By the term “cyclic peptide” is meant a sequence of 5 to 15 amino acids in which the two terminal amino acids are bonded together by a covalent bond which may be a peptide or disulphide bond or a synthetic non-peptide bond such as a thioether, phosphodiester, disiloxane or urethane bond.

By the term “amino acid” is meant an L- or D-amino acid, amino acid analogue or amino acid mimetic which may be naturally occurring or of purely synthetic origin, and may be optically pure, i.e. a single enantiomer and hence chiral, or a mixture of enantiomers. Preferably the amino acids of the present invention are optically pure. By the term “amino acid mimetic” is meant synthetic analogues of naturally occurring amino acids which are isosteres, i.e. have been designed to mimic the steric and electronic structure of the natural compound. Such isosteres are well known to those skilled in the art and include but are not limited to depsipeptides, retro-inverso peptides, thioamides, cycloalkanes or 1,5-disubstituted tetrazoles [see M. Goodman, Biopolymers, 24, 137, (1985)].

When the compound which accumulates at sites of active thrombosis in vivo is a peptide, one or both of the peptide termini may optionally be protected with a suitable metabolism inhibiting group. By the term “metabolism inhibiting group” is meant a biocompatible group which inhibits or suppresses in vivo metabolism of the peptide or amino acid at the amino or carboxyl terminus. Such groups are well known to those skilled in the art and are suitably chosen from, for the peptide amine terminus: acetyl, Boc (where Boc is tert-butyloxycarbonyl), Fmoc (where Fmoc is fluorenylmethoxycarbonyl), benzyloxycarbonyl, trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl). Inhibition of metabolism of the peptide amine terminus may also be achieved by attachment of the amine terminus to a chelating agent when the imaging moiety comprises a metal complex. Suitable metabolism inhibiting groups for the peptide carboxyl terminus include: carboxamide, tert-butyl ester, benzyl ester, cyclohexyl ester, amino alcohol or attachment to a metal complex of a chelating agent. The carboxyl terminus of peptides is particularly susceptible to in vivo cleavage by carboxypeptidase enzymes. Consequently, the carboxyl terminus of the thrombus targeting peptide is preferably protected with a metabolism inhibiting group. Most preferably, both the carboxyl and amino termini of the thrombus targeting peptide are protected with metabolism inhibiting groups. When the peptide comprises a cyclic peptide, the metabolism inhibiting group may be the covalent bond which closes the cyclic peptide ring.

Preferred thrombosis targeting peptides of the present invention are peptide fragments of α₂-antiplasmin. Preferred α₂-antiplasmin peptides of the present invention comprise at least one metabolism inhibiting group, and an amino acid sequence taken from the N-terminus of α₂-antiplasmin, i.e.:

NH₂-Asn-Gln-Glu-Gln-Val-Ser-Pro-Leu-Thr-Leu-Thr-
Leu-Leu-Lys-OH

or variants of this in which one or more amino acids have been exchanged, added or removed such as:

NH₂-Asn-Gln-Glu-Gln-Val-Ser-Pro-Leu-Thr-Leu-Thr-
Leu-Leu-Lys-Gly-OH,
NH₂-Asn-Gln-Glu-Ala-Val-Ser-Pro-Leu-Thr-Leu-Thr-
Leu-Leu-Lys-Gly-OH,
NH₂-Asn-Gln-Glu-Gln-Val-Gly-OH.

Especially preferred α₂-antiplasmin peptides of the present invention are peptide fragments comprising the 4 amino acid sequence Asn-Gln-Glu-Gln (NQEQ). Most especially preferred such α₂-antiplasmin peptides have the sequence:

Asn-Gln-Glu-Gln-Val-Ser-Pro-Xaa-Thr-Leu-Leu-Lys-
Gly,
where
Xaa is Leu, Tyr or I-Tyr (ie. iodo-tyrosine).

Antiplasmin peptides such as the 13-mer NQEQVSPLTLLK can be obtained by the methods described in the literature [Tung et al Chembiochem., 4, 897-899 (2003) and Robinson et al, Circulation, 102, 1151-1157 (2000)].

The α₂-antiplasmin peptides of the present invention preferably have metabolism inhibiting groups attached as described above—preferably at the carboxy terminus, and most preferably at both peptide termini. When a chelator is chosen to be one of the metabolism inhibiting groups, the chelator is preferably attached at the carboxy terminus, and the N-terminus is protected by a metabolism inhibiting group, preferably N-acetyl so that the α₂-antiplasmin peptide is preferably:

[M^{GP}]-Asn-Gln-Glu-Gln-Val-Ser-Pro-Xaa-Thr-Leu-Leu-
Lys-Gly-[chelator]
where:
Xaa is Leu, Tyr or I-Tyr (ie. iodo-tyrosine);
and
M^{GP} is a metabolism inhibiting group.

By the term ‘fluoroalkyl’ is meant an alkyl group with at least one fluorine substituent, ie. the term encompasses groups from monofluoroalkyl (eg. —CH₂F) to perfluoroalkyl (eg. CF₃).

The “imaging moiety” may be detected either external to the human body or via use of detectors designed for use in vivo, such as intravascular radiation or optical detectors such as endoscopes, or radiation detectors designed for intra-operative use. Preferred imaging moieties are those which can be detected externally in a non-invasive manner following administration in vivo. Most preferred imaging moieties are radioactive, especially radioactive metal ions, gamma-emitting radioactive halogens and positron-emitting radioactive non-metals, particularly those suitable for imaging using SPECT or PET.

The “imaging moiety” is preferably chosen from:
(i) a radioactive metal ion;
(ii) a paramagnetic metal ion;
(iii) a gamma-emitting radioactive halogen;
(iv) a positron-emitting radioactive non-metal;
(v) a hyperpolarised NMR-active nucleus;
(vi) a reporter suitable for in vivo optical imaging;
(vii) a β-emitter suitable for intravascular detection.

When the imaging moiety is a radioactive metal ion, ie. a radiometal, suitable radiometals can be either positron emitters such as ⁶⁴Cu, ⁴⁸V, ⁵²Fe, ⁵⁵Co, ^{94m}Tc or ⁶⁸Ga; γ-emitters such as ^{99m}Tc, ¹¹¹In, ^{113m}In, or ⁶⁷Ga. Preferred radiometals are ^{99m}Tc, ⁶⁴Cu, ⁶⁸Ga and ¹¹¹In. Most preferred radiometals are γ-emitters, especially ^{99m}Tc.

When the imaging moiety is a paramagnetic metal ion, suitable such metal ions include: Gd(III), Mn(II), Cu(II), Cr(III), Fe(III), Co(II), Er(II), Ni(II), Eu(III) or Dy(III). Preferred paramagnetic metal ions are Gd(III), Mn(II) and Fe(III), with Gd(III) being especially preferred.

When the imaging moiety is a gamma-emitting radioactive halogen, the radiohalogen is suitably chosen from ¹²³I, ¹³¹I or ⁷⁷Br. A preferred gamma-emitting radioactive halogen is ¹²³I.

When the imaging moiety is a positron-emitting radioactive non-metal, suitable such positron emitters include: ¹¹C, ¹³N, ¹⁵O, ¹⁷F, ¹⁸F, ⁷⁵Br, ⁷⁶Br or ¹²⁴I. Preferred positron-emitting radioactive non-metals are ¹¹C, ¹³N, ¹⁸F and ¹²⁴I, especially ¹¹C and ¹⁸F, most especially ¹⁸F.

When the imaging moiety is a hyperpolarised NMR-active nucleus, such NMR-active nuclei have a non-zero nuclear spin, and include ¹³C, ¹⁵N, ¹⁹F, ²⁹Si and ³¹P. Of these, ¹³C is preferred. By the term “hyperpolarised” is meant enhancement of the degree of polarisation of the NMR-active nucleus over its' equilibrium polarisation. The natural abundance of ¹³C (relative to ¹²C) is about 1%, and suitable ¹³C-labelled compounds are suitably enriched to an abundance of at least 5%, preferably at least 50%, most preferably at least 90% before being hyperpolarised. At least one carbon atom of the compounds which accumulates at sites of active thrombosis in vivo is suitably enriched with ¹³C, which is subsequently hyperpolarised.

When the imaging moiety is a reporter suitable for in vivo optical imaging, the reporter is any moiety capable of detection either directly or indirectly in an optical imaging procedure. The reporter might be a light scatterer (eg. a coloured or uncoloured particle), a light absorber or a light emitter. More preferably the reporter is a dye such as a chromophore or a fluorescent compound. The dye can be any dye that interacts with light in the electromagnetic spectrum with wavelengths from the ultraviolet light to the near infrared. Most preferably the reporter has fluorescent properties.

Preferred organic chromophoric and fluorophoric reporters include groups having an extensive delocalized electron system, eg. cyanines, merocyanines, indocyanines, phthalocyanines, naphthalocyanines, triphenylmethines, porphyrins, pyrilium dyes, thiapyriliup dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include: fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750.

Particularly preferred are dyes which have absorption maxima in the visible or near infrared (NIR) region, between 400 nm and 3 μm, particularly between 600 and 1300 nm. Optical imaging modalities and measurement techniques include, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching. Antiplasmin peptides labelled with a NIR imaging moiety are described by Jaffer et al [Circulation, 110, 170-176 (2004)].

When the imaging moiety is a β-emitter suitable for intravascular detection, suitable such β-emitters include the radiometals ⁶⁷Cu, ⁸⁹Sr, ⁹⁰Y, ¹⁵³Sm, ¹⁸⁶Re, ¹⁸⁸Re or ¹⁹²Ir, and the non-metals ³²P, ³³P, ³⁸S, ³⁸Cl, ³⁹Cl, ⁸²Br and ⁸³Br

When the imaging moiety comprises a metal ion, the metal ion is present as a metal complex. By the term “metal complex” is meant a coordination complex of the metal ion with one or more ligands. It is strongly preferred that the metal complex is “resistant to transchelation”, ie. does not readily undergo ligand exchange with other potentially competing ligands for the metal coordination sites. Potentially competing ligands include the thrombus targeting moiety itself plus other excipients in the preparation in vitro (eg. radioprotectants or antimicrobial preservatives used in the preparation), or endogenous compounds in vivo (eg. glutathione, transferrin or plasma proteins).

Suitable ligands for use in the present invention which form metal complexes resistant to transchelation include: chelating agents, where 2-6, preferably 2-4, metal donor atoms are arranged such that 5- or 6-membered chelate rings result (by having a non-coordinating backbone of either carbon atoms or non-coordinating heteroatoms linking the metal donor atoms); or monodentate ligands which comprise donor atoms which bind strongly to the metal ion, such as isonitriles, phosphines or diazenides. Examples of donor atom types which bind well to metals as part of chelating agents are: amines, thiols, amides, oximes and phosphines. Phosphines form such strong metal complexes that even monodentate or bidentate phosphines form suitable metal complexes. The linear geometry of isonitriles and diazenides is such that they do not lend themselves readily to incorporation into chelating agents, and are hence typically used as monodentate ligands. Examples of suitable isonitriles include simple alkyl isonitriles such as tert-butylisonitrile, and ether-substituted isonitriles such as mibi (i.e. 1-isocyano-2-methoxy-2-methylpropane). Examples of suitable phosphines include Tetrofosmin, and monodentate phosphines such as tris(3-methoxypropyl)phosphine. Examples of suitable diazenides include the HYNIC series of ligands i.e. hydrazine-substituted pyridines or nicotinamides.

Examples of suitable chelating agents for technetium which form metal complexes resistant to transchelation include, but are not limited to:

(i) diaminedioximes of formula:

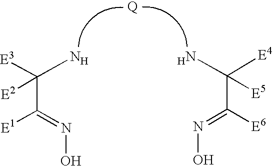

where E¹-E⁶ are each independently an R′ group;
each R′ is H or C₁₋₁₀ alkyl, C₃₋₁₀ alkylaryl, C₂₋₁₀ alkoxyalkyl, C₁₋₁₀ hydroxyalkyl, C₁₋₁₀ fluoroalkyl, C₂₋₁₀ carboxyalkyl or C₁₋₁₀ aminoalkyl, or two or more R′ groups together with the atoms to which they are attached form a carbocyclic, heterocyclic, saturated or unsaturated ring, and wherein one or more of the R′ groups is conjugated to the thrombus targeting compound;
and Q is a bridging group of formula -(J)_{f}-;
where f is 3, 4 or 5 and each J is independently —O—, —NR′— or —C(R′)₂— provided that -(J)_{f}- contains a maximum of one J group which is —O— or —NR′—.

Preferred Q groups are as follows:

Q=—(CH₂)(CHR′)(CH₂)— ie. propyleneamine oxime or PnAO derivatives;
Q=—(CH₂)₂(CHR′)(CH₂)₂— ie. pentyleneamine oxime or PentAO derivatives;

### Q=—(CH₂)₂NR′(CH₂)₂—.

E¹ to E⁶ are preferably chosen from: C₁₋₃ alkyl, alkylaryl alkoxyalkyl, hydroxyalkyl, fluoroalkyl, carboxyalkyl or aminoalkyl. Most preferably, each E¹ to E⁶ group is CH₃.

The thrombus targeting compound is preferably conjugated at either the E¹ or E⁶ R′ group, or an R′ group of the Q moiety. Most preferably, it is conjugated to an R′ group of the Q moiety. When it is conjugated to an R′ group of the Q moiety, the R′ group is preferably at the bridgehead position. In that case, Q is preferably —(CH₂)(CHR′)(CH₂)—, —(CH₂)₂(CHR′)(CH₂)₂— or —(CH₂)₂NR′(CH₂)₂—, most preferably —(CH₂)₂(CHR′)(CH₂)₂—. An especially preferred bifunctional diaminedioxime chelator has the Formula (I):

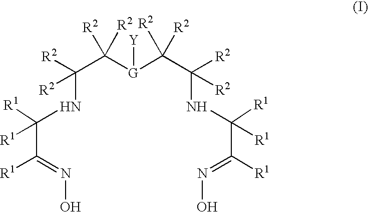

where:
   each R¹ and R² is independently an R group;
   G is N or CR;
   Y is -(A)ₙ-Z
      where: Z is the compound which accumulates at sites of active thrombosis in vivo;
      -(A)ₙ- is a linker group where each A is independently —CO—, —CR₂—, —CR═CR—, —C≡C—, —CR₂CO₂—, —CO₂CR₂—, —NR—NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group or a polyalkyleneglycol, polylactic acid or polyglycolic acid moiety;
      n is an integer of value 0 to 10;
   each R group is independently H or C₁₋₁₀ alkyl, C₃₋₁₀ alkylaryl, C₂₋₁₀ alkoxyalkyl, C₁₋₁₀ hydroxyalkyl, Cl₁₋₁₀ fluoroalkyl, or 2 or more R groups, together with the atoms to which they are attached form a carbocyclic, heterocyclic, saturated or unsaturated ring.

A preferred chelator of Formula (I) is of Formula (Ia):

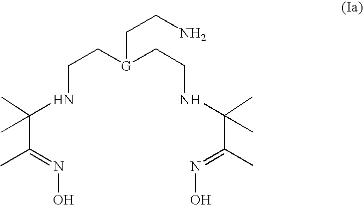

where G is as defined above; such that the thrombus targeting compound is conjugated via the bridgehead —CH₂CH₂NH₂ group.
   (ii) N₃S ligands having a thioltriamide donor set such as MAG₃ (mercaptoacetyltriglycine) and related ligands; or having a diamidepyridinethiol donor set such as Pica;
   (iii) N₂S₂ ligands having a diaminedithiol donor set such as BAT or ECD (i.e. ethylcysteinate dimer), or an amideaminedithiol donor set such as MAMA;
   (iv) N₄ ligands which are open chain or macrocyclic ligands having a tetramine, amidetriamine or diamidediamine donor set, such as cyclam, monoxocyclam or dioxocyclam.
   (v) N₂O₂ ligands having a diaminediphenol donor set.

The above described ligands are particularly suitable for complexing technetium eg. ^{94m}Tc or ^{99m}Tc, and are described more fully by Jurisson et al [Chem. Rev., 99, 2205-2218 (1999)]. The ligands are also useful for other metals, such as copper (⁶⁴Cu or ⁶⁷Cu), vanadium (eg. ⁴⁸V), iron (eg. ⁵²Fe), or cobalt (eg. ⁵⁵Co). Other suitable ligands are described in Sandoz WO 91/01144, which includes ligands which are particularly suitable for indium, yttrium and gadolinium, especially macrocyclic aminocarboxylate and aminophosphonic acid ligands. Ligands which form non-ionic (i.e. neutral) metal complexes of gadolinium are known and are described in U.S. Pat. No. 4,885,363. When the radiometal ion is technetium, the ligand is preferably a chelating agent which is tetradentate. Preferred chelating agents for technetium are the diaminedioximes, or those having an N₂S₂ or N₃S donor set as described above.

It is envisaged that the role of the linker group -(A)ₙ- is to distance the relatively bulky technetium complex which results upon metal coordination, from the active site of the thrombus targeting moiety (Z), so that eg. receptor binding is not impaired. This can be achieved by a combination of flexibility (eg. simple alkyl chains), so that the bulky group has the freedom to position itself away from the active site and/or rigidity such as a cycloalkyl or aryl spacer which orientates the metal complex away from the active site. The nature of the linker group can also be used to modify the biodistribution of the resulting technetium complex of the conjugate. Thus, eg. the introduction of ether groups in the linker will help to minimise plasma protein binding, or the use of polymeric linker groups such as polyalkyleneglycol, especially PEG (polyethyleneglycol) can help to prolong the lifetime of the agent in the blood in vivo.

Preferred linker groups -(A)ₙ- have a backbone chain (ie. the linked atoms which make up the -(A)ₙ- moiety) which contains 2 to 10 atoms, most preferably 2 to 5 atoms, with 2 or 3 atoms being especially preferred. A minimum linker group backbone chain of 2 atoms confers the advantage that the aza-diaminedioxime chelator is well-separated from the biological targeting moiety so that any interaction is minimised. A further advantage is that the potential chelate ring size of the Z group is so large (at least 8 for a 2 atom linker group chain), that these groups are unlikely to compete effectively with the coordination of the chelator to the metal ion. In this way, both the biological targeting characteristics of the thrombus targeting moiety, and the metal complexing capability of the chelator is maintained. It is strongly preferred that the thrombus targeting moiety Z is bound to the chelator in such a way that the linkage does not undergo facile metabolism in blood. That is because such metabolism would result in the imaging metal complex being cleaved off before the labelled biological targeting moiety reaches the desired in vivo target site. The biological targeting moiety is therefore preferably covalently bound to the metal complexes of the present invention via -(A)ₙ- linker groups which are not readily metabolised. Suitable such linkages are carbon-carbon bonds, amide bonds, urea or thiourea linkages, or ether bonds.

Non-peptide linker groups such as alkylene groups or arylene groups have the advantage that there are no significant hydrogen bonding interactions with the conjugated biological targeting moiety so that the linker does not wrap round onto the biological targeting moiety. Preferred alkylene spacer groups are —(CH₂)_{q}— where q is an integer of value 2 to 5. Preferably q is 2 or 3. Preferred arylene spacers are of formula:

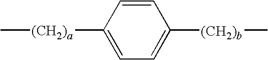

where: a and b are each independently 0, 1 or 2.

A preferred Y group is thus —CH₂CH₂-(A)ₚ-Z, —where p is an integer of value 0 to 3. When Z is a peptide, Y is preferably —CH₂CH₂-(A)ₚ-Z where -(A)ₚ- is —CO— or —NR—. When -(A)ₚ- is —NH—, this grouping has the additional advantage that it stems from the symmetrical intermediate N(CH₂CH₂NH₂)₃, which is commercially available. Triamine precursors having different chain lengths require the use of synthetic strategies to chemically distinguish the various amines (eg. via protecting groups).

The preferred diaminedioxime chelators of Formulae I and Ia are preferably conjugated to the α2-antiplasmin peptides (and preferred embodiments thereof) described above.

When the imaging metal is technetium, the usual technetium starting material is pertechnetate, ie. TcO₄⁻ which is technetium in the Tc(VII) oxidation state. Pertechnetate itself does not readily form metal complexes, hence the preparation of technetium complexes usually requires the addition of a suitable reducing agent such as stannous ion to facilitate complexation by reducing the oxidation state of the technetium to the lower oxidation states, usually Tc(I) to Tc(V). The solvent may be organic or aqueous, or mixtures thereof. When the solvent comprises an organic solvent, the organic solvent is preferably a biocompatible solvent, such as ethanol or DMSO. Preferably the solvent is aqueous, and is most preferably isotonic saline.

When the imaging moiety is radioactive, the thrombus imaging agent is a radiopharmaceutical. Such radiopharmaceuticals are suitably supplied in sterile form suitable for human administration. Such radiopharmaceuticals are suitably supplied in a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 100 cm³ volume) which contains multiple patient doses, whereby single patient doses can thus be withdrawn into clinical grade syringes at various time intervals during the viable lifetime of the preparation to suit the clinical situation.

The radiopharmaceuticals of the present invention may also be supplied in pre-filled syringes. Such pre-filled syringes are designed to contain a single human dose, which is in a form wherein the dose can be administered to the patient directly from the syringe. Such pre-filled syringes are suitably prepared by aseptic manufacture, so that the product is in sterile form. The syringe which is pre-filled is therefore preferably a disposable or other syringe which is suitable for clinical use, and hence which maintains the sterile integrity of the radiopharmaceutical. The pre-filled syringe containing the radiopharmaceutical may advantageously be provided with a syringe shield to protect the operator from radioactive dose. Suitable such radiopharmaceutical syringe shields are known in the art and preferably comprise either lead or tungsten. Such pre-filled syringes are preferably shipped to the customer already fitted with a syringe shield, and packaged within a container which provides further radiation shielding, so that the external radiation dose at the exterior of the package is minimised during transport from the manufacturer to the customer.

A ^{99m}Tc radioactivity content suitable for a ^{99m}Tc diagnostic imaging radiopharmaceutical is in the range 180 to 1500 MBq (3.5 to 42 mCi), depending on the site to be imaged in vivo, the uptake and the target to background ratio. For thrombus imaging with a ^{99m}Tc radiopharmaceutical ca. 740 MBq (20 mCi) would be suitable.

The preparation of the technetium complex composition of the present invention is preferably carried out using a non-radioactive kit, as described in the third embodiment of the invention (below).

The method of the first embodiment preferably further comprises the steps of:
(v) for a patient in which the determination of step (ii) was normal, imaging that individual patient a second time after a period without anticoagulant therapy, with an active thrombus imaging agent as defined above;
(vi) determining from the second image of step (v) whether there is abnormal uptake of the active thrombus imaging agent at any previously known or new locations of VTE relative to venous tissue adjacent to the site of VTE, ie. whether the patient has any recurrence of one or more sites of active VTE;
(vii) resuming the anticoagulant drug therapy if the determination of step (vi) shows that there is recurrence;
(viii) maintaining the cessation of anticoagulant drug therapy if the determination of step (vi) shows that there is no recurrence.

By the term “recurrence of one or more active sites of VTE” is meant recurrence of a site of active thrombosis suggesting continuation of the underlying disease. Such new VTE can occur distal to or locally (ie. at the previously known site).

The second image of step (v) is preferably carried out 5 to 21 days after X, where X is the date of anticoagulant therapy cessation of step (iv) of the method of the present invention. Most preferably, the second image of step (v) is preferably carried out 10 to 14 days after X. Studies in patients with VTE episodes have shown that after cessation of oral anticoagulation therapy there is a rebound period that peaks at approximately 12 days post withdrawal of therapy. The “rebound period” is thus most evident at 10 to 14 days post withdrawal of anticoagulation therapy. This is because the anticoagulant cascade is suppressed during treatment and rebounds during cessation—a process which takes about 12 days. During this time a patient returns to a potentially hypercoaguable state which might result in fresh thrombus formation in a given individual patient. This rebound period is considered critical in the formation of new VTE. Hence, if the imaging agent shows abnormal activity during the rebound period, this indicates a high risk for recurrence and hence the need for further anticoagulation. Steps (v) to (viii) are recommended, since abnormal uptake would signify underlying active thrombosis supporting the decision to continue anticoagulant therapy or conversely if uptake is normal the decision to cease anticoagulant therapy.

In a second aspect, the present invention provides the use of a compound which accumulates at sites of active thrombosis in vivo for the manufacture of an active thrombus imaging agent for use in the method of determination whether or not it is appropriate to cease anticoagulation therapy of the first embodiment.

Preferably, the “compound which accumulates at sites of active thrombosis in vivo” is labelled with an imaging moiety suitable for external imaging of the human body as defined in the first embodiment. The “active thrombus imaging agent” and the “compound which accumulates at sites of active thrombosis in vivo”, including preferred aspects thereof are as defined and described for the first embodiment.

In a third aspect, the present invention provides the use of a kit which comprises the compound which accumulates at sites of active thrombosis in vivo for the manufacture of the active thrombus imaging agent for use in the method of determination whether or not it is appropriate to cease anticoagulation therapy of the first embodiment.

The “compound which accumulates at sites of active thrombosis in vivo” and “active thrombus imaging agent”, including preferred aspects thereof are as defined and described for the first embodiment. The kit provides a convenient form of the compound which accumulates at sites of active thrombus in vivo, and hence use of such a kit is a preferred method of obtaining or preparing the “active thrombus imaging agent”.

Non-radioactive kits for the preparation of radiopharmaceutical compositions are designed to give sterile radiopharmaceutical products suitable for human administration, e.g. via direct injection into the bloodstream. For ^{99m}Tc, the kit is preferably lyophilised and is designed to be reconstituted with sterile ^{99m}Tc-pertechnetate (TcO₄⁻) from a ^{99m}Tc radioisotope generator to give a solution suitable for human administration without further manipulation. Suitable kits comprise a container (eg. a septum-sealed vial) containing the uncomplexed chelating agent, together with a pharmaceutically acceptable reducing agent such as sodium dithionite, sodium bisulphite, ascorbic acid, formamidine sulphinic acid, stannous ion, Fe(II) or Cu(I); together with at least one salt of a weak organic acid with a biocompatible cation. By the term “biocompatible cation” is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

The non-radioactive kits may optionally further comprise a second, weak organic acid or salt thereof with a biocompatible cation, which functions as a transchelator. The transchelator is a compound which reacts rapidly to form a weak complex with technetium, then is displaced by the chelator of the kit. This minimises the risk of formation of reduced hydrolysed technetium (RHT) due to rapid reduction of pertechnetate competing with technetium complexation. Suitable such transchelators are the weak organic acids and salts thereof described above, preferably tartrates, gluconates, glucoheptonates, benzoates, or phosphonates, preferably phosphonates, most especially diphosphonates. A preferred such transchelator is MDP, ie. methylenediphosphonic acid, or a salt thereof with a biocompatible cation.

As an alternative to use of the chelator in free form, the kit may optionally contain a non-radioactive metal complex of the chelator which, upon addition of the technetium, undergoes transmetallation (i.e. ligand exchange) giving the desired product. Suitable such complexes for transmetallation are copper or zinc complexes.

The pharmaceutically acceptable reducing agent used in the kit is preferably a stannous salt such as stannous chloride, stannous fluoride or stannous tartrate, and may be in either anhydrous or hydrated form. The stannous salt is preferably stannous chloride or stannous fluoride.

The non-radioactive kits may optionally further comprise additional components such as a radioprotectant, antimicrobial preservative, pH-adjusting agent, or filler. By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (ie. 4-aminobenzoic acid), gentisic acid (ie. 2,5-dihydroxybenzoic acid) and salts thereof with a biocompatible cation. By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the radiopharmaceutical composition post-reconstitution, ie. in the radioactive diagnostic product itself. The antimicrobial preservative may, however, also optionally be used to inhibit the growth of potentially harmful micro-organisms in one or more components of the non-radioactive kit of the present invention prior to reconstitution. Suitable antimicrobial preservative(s) include: the parabens, ie. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds used to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], and pharmaceutically acceptable bases such as sodium carbonate, sodium bicarbonate or mixtures thereof.

When the biological targeting moiety is a peptide fragment of α₂-antiplasmin, a preferred kit formulation comprises: the ligand of Formula (I) or (Ia), stannous reductant, an acetate salt of a biocompatible cation, a diphosphonic acid transchelator plus a pH-adjusting agent. A preferred such kit comprises: the ligand of Formula (Ia) where G is N; stannous chloride; sodium acetate; MDP or a biocompatible salt thereof; a radioprotectant, especially PABA or a biocompatible salt thereof, most especially the sodium salt of PABA; and sodium bicarbonate as the pH-adjusting agent. A most preferred such kit further comprises the ligand of Formula I, where each R¹ is CH₃, (A)ₚ is NH and Z is Ac-Asn-Gln-Glu-Gln-Val-Ser-Pro-Xaa-Thr-Leu-Leu-Lys-Gly-, where Xaa is Tyr or I-Tyr, and Ac is N-acetyl.

The invention is illustrated by the non-limiting Examples detailed below. Examples 1 to 3 provide the synthesis of chelator conjugate of an antiplasmin peptide of the present invention. Example 4 shows the ^{99m}Tc radiolabelling of this peptide. Example 5 provides a lyophilised kit, which is further described in WO 04/037297. Example 6 provides the preparation of ^{99m}Tc-labelled Compound 3 from such a kit. Example 7 describes the results of a clinical trial in VTE patients, in which an expert panel analysis of the images concluded that the agent differentiated between active and inactive VTE.

TABLE 1

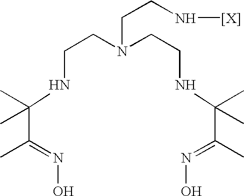

| Compound | X |
|---|---|
| 1. | H |
| 3. | -GKLLT(I-Y)PSVQEQN-Ac |

Where: Ac is acetyl.

### EXAMPLE 1

### Synthesis of 3,3,11,11-tetramethyl-7-(2-aminoethyl)-4,7,10-triazatridecane-2,12-dionedioxime (Compound 1 or Pn216)

To a solution of tris(2-aminoethyl)amine (1 ml, 6.68 mmol) in acetonitrile (10 ml) was added sodium bicarbonate (1.12 g, 13.36 mmol, 2 eq). A solution of 3-chloro-3-methyl-2-nitrosobutane [R. K. Murmann, J. Am. Chem. Soc, 79, 521-526 (1957); 1.359 g, 10.02 mmol, 1.5 eq] in dry acetonitrile (5 ml) was added slowly. The reaction mixture was left to stir at room temperature for 3 days, and then filtered. The residue was washed well with acetonitrile, and the filtrate evaporated. The crude product was then purified by RP-HPLC (column: Hamilton PRP-1; gradient: 0 to 100% B in 20 min; where Eluent A is 2% aqueous NH₃ and Eluent B is acetonitrile, at a flow rate of 3 ml/min) to afford Compound 1 (164 mg, 7%).

δ_{H} (CD₃OD, 300 MHz): 2.77 (2H, t, J=6 Hz, CH₂NH₂), 2.50-2.58 (10H, m, H₂NCH₂CH₂N(CH₂CH₂NH)₂), 1.85 (6H, s, 2×CH₃C═N), 1.23 (12H, s, 2×(CH₃)₂CNH).

### EXAMPLE 2

### Synthesis of the Peptide Ac-NQEQVSPY(3I)TLLKG (Compound 2)

The protected peptide Ac-Asn(Trt)-Gln(Trt)-Glu(OtBu)-Gln(Trt)-Val-Ser(tBu)-Pro-Tyr(3I)-Thr(tBu)-Leu-Leu-Lys(Boc)-Gly-OH was assembled on a 2-chlorotrityl resin by anchoring Fmoc-Gly- to the resin, and then successive deprotections/coupling cycles with the appropriate protected amino acids and the coupling reagents DCC and HOBt. Solid phase peptide synthesis is described in as described in P. Lloyd-Williams, F. Albericio and E. Girald; Chemical Approaches to the Synthesis of Peptides and Proteins, CRC Press, 1997. The terminal asparagine was acetylated, cleaved from the resin using 0.5% TFA and Compound 2 used without further purification as the trifluoroacetate salt.

### EXAMPLE 3

### Synthesis of Compound 3

The protected Ac-NQEQVSPY(3I)TLLKG peptide (Compound 2) was cleaved from the solid phase resin as described in Example 2, and then coupled with Compound 1 in solution using PyBOP (benzotriazole-1-yl-oxytris-pyrrolidino-phosphonium hexafluorophosphate) and HOBt (1-hydroxybenzotriazole) as the coupling agents. Compound 3 was obtained by deprotection in reagent K [reagent K is 82.5% TFA, 5% phenol, 5% processed water, 5% thioanisole, 2.5% ethanedithiol (EDT)]. The crude conjugate was first purified by RP-HPLC using TFA followed by a second purification and salt exchange with acetic acid, lyophilisation, filtration with a 0.22μ filter and a final lyophilisation to give Compound 3.

Molecular weight by MS 1970±1 Daltons.

### EXAMPLE 4

### Tc-99m Radiolabelling of Compound 3

A 0.1 ml aliquot of Compound 3 dissolved in H₂O (1 mg/ml) was transferred to a nitrogen-filled 10 ml glass vial together with deoxygenated saline (0.9% w/v, 1 ml) and 0.035 ml aqueous NaOH (0.1M). To this solution was added ^{99m}Tc technetium generator eluate (1 ml, approx. 0.4 GBq) and then aqueous stannous chloride solution (0.1 ml, ca. 10 μg). The labelling pH was 9.0-10.0. Vials were incubated at ambient laboratory temperature (15-25° C.) for 30 minutes to effect labelling. The resulting preparation was either diluted to the desired radioactive concentration or HPLC purification was performed (System B) to remove unlabelled starting material and radioactive impurities prior to testing. After purification the organic solvent was removed in vacuo and the sample was redissolved in about 5 ml 0.1M phosphate buffer pH 7.4 to give a working concentration of 6-9 MBq/ml. Radiochemical purity was assessed before use by the thin layer chromatography (TLC) system described below:

i) ITLC SG 2 cm×20 cm eluted with 0.9% w/v saline
ii) Whatman No. 1 2 cm×20 cm eluted with 50:50 v/v acetonitrile: H₂O.
The labelled Compound 3 remains at, or close to, the origin in TLC system (i) and moves close to the solvent front in system (ii).

HPLC System B.

| | |
|---|---|
| Flow Rate: | 1 ml/min and TFA = trifluoroacetic acid. |
| Column | Waters Novapak C18 150 × 3.9 mm. |
| | Particle size 4 microns |
| Gradient: | Elution Profile 0-100% B in 22 min. |
| Eluent A: | 0.1% aqueous TFA |
| Eluent B: | 0.1% TFA in acetonitrile |

### EXAMPLE 5

### Preparation of Lyophilised Kits

Approximately 90% of the total volume of water for injection (WFI) was placed in the preparation vessel and deoxygenated by nitrogen purging. Methylene diphosphonic acid, stannous chloride dihydrate, Compound 3 (as the acetate salt), sodium acetate trihydrate and p-aminobenzoic acid, sodium salt were added in turn allowing each one to dissolve whilst nitrogen purging was continued. The nitrogen purging of the solution was replaced with a nitrogen flow over the solution headspace. Sodium hydrogen carbonate and sodium carbonate anhydrous, or sodium hydrogen carbonate alone were then added, and allowed to dissolve. The bulk solution was then adjusted to 100% of the final volume (˜5 litre) with deoxygenated WFI. The bulk solution was then filtered through two sterile 0.2 μm filters into the filling vessel. The headspace of the filling vessel was purged with sterile filtered (0.2 μm) nitrogen or argon for the duration of filling operation. Aliquots of 1.0 ml were dispensed aseptically into vials. The vials were half-stoppered with closures A or C and transferred onto pre-chilled freeze-dryer shelves. The vials were then lyophilised, back-filled with sterile filtered (0.2 μm) nitrogen gas, stoppered and sealed.

TABLE 2
Kit formulations.

| | Cmpd 3 | SnCl₂•2H₂O | MDP | NaHCO₃ | Na₂CO₃ | NaOAc•3H₂O | Na-pABA | |
|---|---|---|---|---|---|---|---|---|
| Lot | (μg) | (μg) | (μg) | (μg) | (μg) | (μg) | (μg) | Closure |
| 82/4 | 100 | 36 | 90 | 1320 | 98 | 6600 | 50 | A |
| 82/6 | 100 | 36 | 90 | 3300 | 0 | 530 | 200 | C |

where Closure A = PH701/45 AS red-brown; C = 4432/50 1178 grey.

Formulation 82/4 was used in the human studies of Example 7.

### EXAMPLE 6

### Preparation of ^{99m}Tc-Labelled Compound 3 from Lyophilised Kits

The freeze-dried kits of Example 6 were reconstituted with ^{99m}Tc-pertechnetate (2-8 ml of technetium generator eluate; 0.5-2.5 GBq). The solution was heated in a water bath or appropriate vial heater at 60-65° C. for 10-12 minutes. Radiochemical purity was determined by HPLC and the RHT was determined by the ITLC method described below:

ITLC SG 2 cm×20 cm eluted with a 50:50 (v/v) methanol: ammonium acetate solution (1M). The ligand based radiolabelled species move to the solvent front. RHT is retained at the origin.

### EXAMPLE 7

### Imaging in Human Patients

^{99m}Tc-Labelled Compound 3 was prepared from a kit as described in Examples 5 and 6. The agent was studied in 2 randomised clinical trials of 30 and 50 human patients. In the first human patient trial 30 subjects with confirmed PE (by spiral CT) were included, and the sCT images compared with those obtained after the injection of the ^{99m}Tc-labelled compound. In the second human patient trial a total of 50 individuals with clinical suspicion of PE were recruited. These individuals underwent commonly prescribed imaging tests to detect the presence/absence of PE and DVT (i.e. perfusion/ventilation lung scans, CT scan, ultrasound of legs). The results from these tests were analysed by an expert panel along with all relevant clinical information to make a final diagnosis of PE present/absent. The results and images from the trial were reviewed by a panel of experts in the field of VTE.

It was found that labelling with the agent was most apparent in clots which had been newly formed or were still active due to continuing underlying pathology (based on imaging comparison with CT scan images). Also, when the agent was not taken up in pulmonary emboli previously detected on a CT scan (CT angiography or CTA), those emboli exhibited the anatomical characteristics of organised inactive thrombosis via CTA. Images of the thorax and lower extremities of patients with PE or DVT where abnormal uptake was apparent were compared with the equivalent CTA images. Similarly, patients who had been diagnosed with the above conditions but showed no abnormal uptake in the thorax despite the diagnosis, were also compared with the equivalent CTA images. Whole body scans with the agent also showed some patients with no abnormal uptake in the lungs but who still had abnormal uptake in the lower extremities. There were also patients who whilst not having a PE did show abnormal uptake in the pleural cavity in some cases and not in others. The CT scans of these patients showed the presence of pleural effusion irrespective of whether the agent's uptake was abnormal or not. The CT scan images were able to identify the presence of clots in the lungs of patients with PE and were used as the standard of truth.

The conclusions from the expert panel are summarised as follows:
(i) abnormal uptake with the agent was apparent in thrombus which was newly formed (based on the CT scan);
(ii) no abnormal uptake in the lungs was observed in thrombus which was older and resolving (based on the equivalent CT scan image of the thrombus). In some of these patients, abnormal uptake was noted in the lower extremities consistent with an ongoing active DVT;
(iii) in patients where there was no evidence of a pulmonary embolism based on the CT scan, abnormal uptake in the lung with the agent was observed. It was concluded that the abnormal uptake was identifying a site of localised inflammation associated with extracellular cross-linked fibrin. The patient's clinical history suggested an ongoing chronic obstructive pulmonary disease (COPD), a clinical condition of active lung inflammation with possible cross-linked fibrin deposition. This observation was consistent with the patient's medical history;
(iv) the CT scan demonstrated pleural effusion in two cases. In the first case, abnormal uptake was observed with the agent. The patient's underlying medical condition suggested the presence of infection, thus the agent was able to identify the presence of an exudates (comprising inflammatory mediators and cells and cross linked extracellular fibrin). In the second case, the CT scan again showed pleural effusion, but there was no associated abnormal uptake with the agent. The patient's medical history showed the patient had chronic heart and renal failure which can lead to the presence of a transudate in the pleural cavity (a watery extracellular fluid). Since a transudate does not involve inflammatory mediators or cross-linked fibrin, the imaging agent is not able to identify such a condition. In both these patients the conclusion was that the imaging agent had been able to differentiate between these two conditions (the CT scan could not) and is consistent with its mechanism of action;
(v) the imaging agent is able to identify areas of inflammatory activity associated with cross-linked fibrin and is able to differentiate between active and inactive or resolving thrombus. In venous thromboembolism, recurrence of the condition usually is manifested by a renewed site of a deep vein thrombosis. The agent was able to identify sites of DVT very effectively as well as active thrombus caused by a recent pulmonary embolism. A pulmonary embolism is typically caused by the breaking of thrombus from an existing DVT.

The panel concluded that ^{99m}Tc-Labelled Compound 3 is able to differentiate between active and inactive VTE disease and that this characteristic can be used to support a decision to continue or cease anticoagulant therapy.

What is claimed is:

1. A method of determining whether or not it is appropriate to cease anticoagulation therapy for an individual patient previously diagnosed with venous thrombo-embolism (VTE) who is considered to be potentially ready for cessation of anticoagulant therapy, which method comprises:
(i) imaging said patient with a thrombus imaging agent which comprises a compound which accumulates at sites of actively forming thrombosis in vivo labelled with an imaging moiety suitable for external imaging of the human body;
(ii) making a determination from the imaging of step (i) whether there is abnormal uptake of the thrombus imaging agent at the known locations of VTE relative to venous tissue adjacent to the site of VTE;
(iii) when the determination of step (ii) shows abnormal uptake, that site of thrombosis is identified as being active and the anticoagulant drug therapy for that patient is continued;
(iv) when the determination of step (ii) is normal, that site of thrombosis is identified as being inactive and the anticoagulant drug therapy for that patient is ceased.

2. The method of claim 1, where the VTE is acute idiopathic VTE.

3. The method of claim 1, where the VTE is deep vein thrombosis (DVT) or pulmonary embolism (PE).

4. The method of claim 1, where the imaging moiety comprises a radioisotope, MRI contrast agent or near-IR (NIR) optical imaging dye.

5. The method of claim 1, where the compound which accumulates at sites of active thrombosis in vivo is chosen from:
(a) a 5 to 30 mer peptide fragment of antiplasmin;
(b) a fibrin-binding peptide based on the fibrin binding domain of fibronectin;
(c) an agent which targets the IIIa/IIb receptor associated with sensitised platelets.

6. The method of claim 5, where the 5 to 30 mer peptide fragment of antiplasmin comprises the peptide sequence NQEQ.

7. The method of claim 1, where the imaging is carried out on the patient's legs.

8. The method of claim 1, where the active thrombosis imaging agent is prepared from a kit which comprises the compound which accumulates at sites of active thrombosis in vivo.

9. The method of claim 1, where the anticoagulation therapy comprises drug medication chosen from: the intravenous agents heparin or low molecular weight fractionated heparin, or oral agents chosen from a vitamin K inhibitor or antagonist.

10. The method of claim 9, where the vitamin K inhibitor or antagonist comprises warfarin.

11. The method of claim 1, further comprising the steps of:
(v) for a patient in which the determination of step (ii) of claim 1 was normal, imaging that individual patient a second time after a period without anticoagulant therapy, with an active thrombosis imaging agent as defined in claims 1 to 8;
(vi) determining from the second image of step (v) whether there is abnormal uptake of the active thrombus imaging agent at any previously known or new locations of VTE relative to venous tissue adjacent to the site of VTE, ie. whether the patient has any recurrence of one or more sites of active VTE;
(vii) resuming the anticoagulant drug therapy if the determination of step (vi) shows that there is recurrence;
(viii) maintaining the cessation of anticoagulant drug therapy if the determination of step (vi) shows that there is no recurrence.

12. The method of claim 11, wherein the second imaging of step (v) is carried out 10 to 14 days after X further wherein said method comprises
(i) imaging said patient with a thrombus imaging agent which comprises a compound which accumulates at sites of actively forming thrombosis in vivo labelled with an imaging moiety suitable for external imaging of the human body;
(ii) making a determination from the imaging of step (i) whether there is abnormal uptake of the thrombus imaging agent at the known locations of VTE relative to venous tissue adjacent to the site of VTE;
(iii) when the determination of step (ii) shows abnormal uptake that site of thrombosis is identified as being active and the anticoagulant drug therapy for that patient is continued;
(iv) when the determination of step (ii) is normal, that site of thrombosis is identified as being inactive and the anticoagulant drug therapy for that patient is ceased and where X is the date of anticoagulant therapy cessation of step (iv).

13. The method of claim 11, where the active thrombosis imaging agent of step (v) is the same as that used for imaging said patient with a thrombus imaging agent which comprises a compound which accumulates at sites of actively forming thrombosis in vivo labelled with an imaging moiety suitable for external imaging of the human body.

14. The method of claim 1, wherein a compound which accumulates at sites of active thrombosis in vivo for the manufacture of an active thrombus imaging agent is used to determine whether it is appropriate to cease anticoagulation therapy of said claim.

15. The method of claim 14, where the compound which accumulates at sites of active thrombosis in vivo is part of a kit.

16. The method of claim 14, where the compound which accumulates at sites of active thrombosis in vivo is labelled with an imaging moiety suitable for external imaging of the human body wherein said labeling comprises
(i) imaging said patient with a thrombus imaging agent which comprises a compound which accumulates at sites of actively forming thrombosis in vivo labelled with an imaging moiety suitable for external imaging of the human body;
(ii) making a determination from the imaging of step (i) whether there is abnormal uptake of the thrombus imaging agent at the known locations of VTE relative to venous tissue adjacent to the site of VTE;
(iii) when the determination of step (ii) shows abnormal uptake, that site of thrombosis is identified as being active and the anticoagulant drug therapy for that patient is continued;
(iv) when the determination of step (ii) is normal, that site of thrombosis is identified as being inactive and the anticoagulant drug therapy for that patient is ceased.

17. The method according to claim 14, where the compound which accumulates at sites of active thrombosis in vivo is chosen from
(a) a 5 to 30 mer peptide fragment of antiplasmin;
(b) a fibrin-binding peptide based on the fibrin binding domain of fibronectin;
(c) an agent which targets the IIIa/IIb receptor associated with sensitised platelets.

18. (canceled)

19. (canceled)
